(19) United States
(12) Patent Application Publication
(10) Pub. No.: US 2008/0176955 A1
(43) Pub. Date: Jul. 24, 2008

(54) COMBINED ADMINISTRATION OF BENZONATATE AND GUAIFENESIN

(75) Inventors: Thomas Heck, Holly Springs, NC (US); Michael Cvijanovich, Apex, NC (US)

Correspondence Address: WILSON SONSINI GOODRICH & ROSATI, 650 PAGE MILL ROAD, PALO ALTO, CA, 94304-1050, US

(73) Assignee: VICTORY PHARMA, INC., San Diego, CA (US)

(21) Appl. No.: 12/015,413

(22) Filed: Jan. 16, 2008

Related U.S. Application Data
(60) Provisional application No. 60/885,154, filed on Jan. 16, 2007.

(51) Int. Cl. A61K 31/085; A61P 11/00

(52) U.S. Cl. 514/716

(57) ABSTRACT

Pharmaceutical compositions are provided containing a combination of benzonatate and guaifenesin, or pharmaceutically acceptable salts thereof. Methods of using compositions comprising benzonatate and guaifenesin, or pharmaceutically acceptable salts thereof, provide relief from cough, pulmonary congestion or both. The compositions and methods provide relief to opiate-sensitive individuals, in particular, as well as to infants and other pediatric patients.

### PRIORITY CLAIM AND RELATED UNITED STATES APPLICATION

This application claims priority from U.S. provisional patent application 60/885,154, filed Jan. 16, 2007, which is incorporated herein by reference in its entirety.

### BACKGROUND OF THE INVENTION

Coughs can vary in severity from mildly annoying to extremely painful and harmful. Coughing may be caused by colds, bronchitis, lung congestion, allergy, post nasal drip, pneumonia and other respiratory maladies. Cough caused by the common cold can be very persistent and severe coughing can cause intense discomfort and injury to the patient, in some cases resulting in broken, bruised, strained and separated ribs. Even mild coughs can disrupt a patient's well-being, in some cases making it difficult or impossible to sleep at night or concentrate during the day. Thus, cough suppression is often an important part of therapy for all sorts of respiratory illnesses.

One of the earliest-used classes of medicaments for the treatment of cough comprised the opioids, such as codeine. Dextromethorphan (3-methoxy-17-methyl-9(α),13(α),14(α)-morphinan hydrobromide monohydrate) was developed as a non-narcotic substitute for codeine, as it lacks the physically addictive properties of codeine at antitussive doses, although it is still prone to abuse and can lead to psychological addiction. Doses of seven to 50 times the generally prescribed dose of dextromethorphan can cause dissociation and other altered psychological states. Other non-narcotic antitussives, such as benzonatate, have been developed subsequent to the introduction of dextromethorphan.

While cough suppression is considered desirable, and in some cases necessary, a productive cough (a cough causing expulsion of mucus, of phlegm) does serve the salutary purpose of clearing phlegm from the respiratory tract, and in particular from the lungs. The collection of phlegm in the lungs may lead to cause, exacerbate or inhibit recovery from pneumonia. Moreover, unchecked buildup of phlegm in the lungs can lead to decreased oxygen-carbon dioxide exchange across the pulmonary epithelium, reducing the oxygen saturation of the blood and thereby causing anoxia. In order to aid the clearance of pulmonary phlegm, patients suffering from coughs are often given expectorants, which, as implied by their name, enhance the ability to eliminate phlegm from the body (e.g. by expectoration). It is thus common practice to administer an expectorant, such as guaifenesin, to a patient having a productive cough (i.e. one caused by excessive respiratory tract mucus), either with or in the absence of dextromethorphan.

While it is known to combine dextromethorphan with an expectorant, such as guaifenesin, for antitussive therapy, it is not known to combine benzonatate and guaifenesin in a single dosage form for administration to a patient.

The present invention addresses the foregoing problem and provides related advantages as well.

### SUMMARY

The present invention addresses the foregoing problem and provides related advantages by providing, in some embodiments, a pharmaceutical composition comprising a therapeutically effective combination of benzonatate, or a pharmaceutically acceptable salt thereof, and guaifenesin. In some embodiments, the pharmaceutical composition is an orally available unit dosage form, while in other embodiments, the pharmaceutical composition is an oral capsule or gel capsule. In some embodiments, the pharmaceutical composition is adapted to provide a dose of about 50 mg to about 400 mg of benzonatate and about 100 mg to about 600 mg of guaifenesin per dose. In some embodiments, the pharmaceutical composition provides approximately 100 mg to 200 mg of benzonatate and about 200 mg to about 400 mg of guaifenesin per dose. The dose may be split between multiple discrete dosage units, such as tablets, caplets, capsules, gel caps, etc. In some embodiments, each discrete dosage unit contains about 100 mg of benzonatate and about 400 mg of guaifenesin. In particular embodiments, the patient takes or is given 1 to 4, preferably 1 or 2 such units containing about 100 mg of benzonatate and about 400 mg of guaifenesin as needed, not to exceed 4 doses per day. Thus, in some embodiments, there is provided a dosage form capable of delivering a daily dosage of about 400 mg to about 800 mg of benzonatate and about 1200 mg to about 2400 mg of guaifenesin per day in one to four doses per day. In some currently preferred embodiments, the preferred dosing regimen is once daily; in other currently preferred embodiments, the preferred dosing regimen is twice daily.

In some embodiments, the invention provides a method of treating a cough, comprising co-administering to a patient a therapeutically effective amount of a composition comprising benzonatate, or a pharmaceutically acceptable salt thereof, and guaifenesin. In some embodiments, the method comprises providing benzonatate, or a pharmaceutically acceptable salt thereof, and guaifenesin in an oral dosage form. In some embodiments, the oral dosage form is an oral tablet, caplet, capsule or gel cap. In some embodiments, the method comprises administering a dose of about 50 mg to about 400 mg of benzonatate and about 100 mg to about 600 mg of guaifenesin per dose to a patient suffering from coughing. In some embodiments, the pharmaceutical composition provides approximately 100 mg to 200 mg of benzonatate and about 200 mg to about 400 mg of guaifenesin per dose. The dose may be split between multiple discrete dosage units, such as tablets, caplets, capsules, gel caps, etc. In some embodiments, each discrete dosage unit contains about 100 mg of benzonatate and about 200 mg of guaifenesin. In particular embodiments, the patient takes or is given 1 to 4, preferably 1 or 2 such units containing about 100 mg of benzonatate and about 400 mg of guaifenesin as needed, not to exceed 4 doses per day. Thus, in some embodiments, there is provided a method of treating cough with a dosage form capable of delivering a daily dosage of about 200 mg to about 800 mg of benzonatate and about 1200 mg to about 2400 mg of guaifenesin per day in one to four doses per day. In some embodiments, patient is a pediatric patient. In some currently preferred embodiments, the preferred dosing regimen is once daily; in other currently preferred embodiments, the preferred dosing regimen is twice daily.

The foregoing and other advantages are further provided by embodiments of the invention, which provide a method of treating congestion of the lung, comprising co-administering to a patient a therapeutically effective amount of a composition comprising benzonatate, or a pharmaceutically acceptable salt thereof, and guaifenesin or a pharmaceutically acceptable salt thereof. In some embodiments, the pharmaceutical composition comprises benzonatate, or a pharmaceutically acceptable salt thereof, and guaifenesin, or a pharmaceutically acceptable salt thereof, is administered in an oral dosage form. In some embodiments, the oral dosage form is an oral capsule. In some embodiments, the pharmaceutical composition is adapted to provide a dose of about 50 mg to about 400 mg of benzonatate and about 100 mg to about 600 mg of guaifenesin per dose. In some embodiments, the pharmaceutical composition provides approximately 100 mg to 200 mg of benzonatate and about 200 mg to about 400 mg of guaifenesin per dose. The dose may be split between multiple discrete dosage units, such as tablets, caplets, capsules, gel caps, etc. In some embodiments, each discrete dosage unit contains about 100 mg of benzonatate and about 400 mg of guaifenesin. In particular embodiments, the patient takes or is given 1 to 4, preferably 1 or 2 such units containing about 100 mg of benzonatate and about 400 mg of guaifenesin as needed, not to exceed 4 doses per day. In some embodiments, the invention provides a dosage form capable of delivering a daily dosage of about 200 mg to about 800 mg of benzonatate and about 1200 mg to about 2400 mg of guaifenesin per day in one to four doses per day. In some currently preferred embodiments, the preferred dosing regimen is once daily; in other currently preferred embodiments, the preferred dosing regimen is twice daily.

Moreover, the foregoing and additional advantages are provided by embodiments of the invention, which provide a method of simultaneously treating lung congestion and cough, comprising administering to a patient a therapeutically effective amount of a composition comprising benzonatate and guaifenesin. In some embodiments, the composition comprises benzonatate, or a pharmaceutically acceptable salt thereof, and guaifenesin, or a pharmaceutically acceptable salt thereof, is administered in an oral dosage form. In some embodiments, the oral dosage form is an oral capsule. In some embodiments, the pharmaceutical composition is adapted to provide a dose of about 50 mg to about 400 mg of benzonatate and about 100 mg to about 600 mg of guaifenesin per dose. In some embodiments, the pharmaceutical composition provides approximately 100 mg to 200 mg of benzonatate and about 200 mg to about 400 mg of guaifenesin per dose. The dose may be split between multiple discrete dosage units, such as tablets, caplets, capsules, gel caps, etc. In some embodiments, each discrete dosage unit contains about 100 mg of benzonatate and about 200 mg of guaifenesin. In particular embodiments, the patient takes or is given 1 to 4, preferably 1 or 2 such units containing about 100 mg of benzonatate and about 400 mg of guaifenesin as needed, not to exceed 4 doses per day. Thus, in some embodiments, there is provided a dosage form capable of delivering a daily dosage of about 200 mg to about 800 mg of benzonatate and about 1200 mg to about 2400 mg of guaifenesin per day in one to four doses per day. In some embodiments, the patient is a pediatric patient. In some currently preferred embodiments, the preferred dosing regimen is once daily; in other currently preferred embodiments, the preferred dosing regimen is twice daily.

Additional advantages are provided by embodiments of the invention, which provide a therapeutically effective combination of benzonatate, or a pharmaceutically acceptable salt thereof, and guaifenesin in a kit form. In some embodiments, the kit comprises a first dosage unit that comprises benzonatate, or a pharmaceutically acceptable salt thereof, and a second dosage unit that comprises guaifenesin, wherein combined consumption of the first and second dosage units provides a therapeutically effective dose of a combination of benzonatate and guaifenesin to the patient. In some particular embodiments, the first dosage unit is substantially free of guaifenesin. In some particular embodiments, the second dosage unit is substantially free of benzonatate. In some currently preferred embodiments, the first dosage unit contains no guaifenesin and the second dosage unit contains no benzonatate. In some particular embodiments of the invention, the first dosage unit is an oral dosage unit adapted to resist disintegration in the mouth. In some currently preferred embodiments, the preferred dosing regimen is once daily; in other currently preferred embodiments, the preferred dosing regimen is twice daily.

These and additional embodiments, advantages and characteristics are provided by embodiments of the invention as provided herein.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

In some embodiments, the invention provides a pharmaceutical composition comprising a therapeutically effective combination of benzonatate, or a pharmaceutically acceptable salt thereof, and guaifenesin or a pharmaceutically acceptable salt thereof. In some embodiments, the pharmaceutical composition is an orally available unit dosage form. In some embodiments, the pharmaceutical composition is an oral capsule. In some embodiments, the pharmaceutical composition is adapted to provide a dose of about 50 mg to about 400 mg of benzonatate and about 100 mg to about 600 mg of guaifenesin per dose.

Benzonatate

Benzonatate (2,5,8,11,14,17,20,23,26-nonaoxaoctacosan-28-yl parabutylaminobenzoate) is represented by the structural formula:

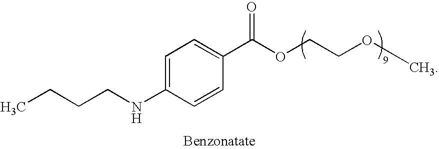

Benzonatate is a non-narcotic oral antitussive drug, which works by a different mechanism than the opioid antitussive drugs and dextromethorphan. While the latter crosses the blood-brain barrier, acting centrally to raise the tussive threshold, benzonatate works by anesthetizing the tissues of the lungs and pleura that are responsible for the cough reflex. Indeed its antitussive action appears to arise from its ability to anesthetize the pulmonary stretch sensors, which, when stretched during breathing, cause a cough.

When administered in a discrete dosage unit and/or when co-administered with guaifenesin (and/or other ingredients), benzonatate may be administered by any suitable route of administration, although oral administration is currently preferred. Conveniently, benzonatate may be administered in oral form, e.g. as 1 or more oral tablets, capsules, caplets or gel caps. It is noted that benzonatate's local anesthetic properties require that it be administered in a dosage unit that keeps benzonatate within the unit until it has entered the stomach or intestines. Thus, in some embodiments of the invention, tablets and caplets containing benzonatate alone or in combination with guaifenesin are coated with a suitable coating that prevents disintegration of the dosage unit in the mouth. In other embodiments, the invention provides capsules or gel caps containing benzonatate (alone or in combination with guaifenesin), which by their nature resist disintegration in the mouth. In particular embodiments, benzonatate (alone or in combination with guaifenesin) may be administered as an oral extended release tablet, which by its nature also provides protection from oral disintegration of the dosage unit. In other embodiments, benzonatate may be administered as an oral immediate release formulation. In such embodiments, the dosage unit is coated with a coating, or encapsulated within a capsule or gel cap, which resists oral disintegration but immediately releases the contents of the dosage unit upon contact with the stomach. In some embodiments, immediate release coatings or encapsulating agents are pH sensitive, dissolving slowly at neutral pH but very rapidly at the pH of the stomach, e.g. about pH 1. Such coatings and encapsulating materials are known to the person of skill in the art.

In embodiments according to the invention, the dosage of benzonatate is in the range of about 5 to about 1000 mg/day. The dosage may be given all at once or, as is currently preferred, may be divided amongst 2 or more doses. For example, the daily dose may be divided amongst 2 to 6, preferably about 3, doses of from 50 mg/dose to about 200 mg/dose, preferably about 100 mg/dose. In the case of pediatric patients, e.g. patients under the age of 12, the dose is reduced to about 1 to about 400 mg/day, especially about 5 mg/day to about 200 mg/day, and in particular about 10 mg/dose to about 30 mg/dose administered 2 to 6, preferably about 3, times per day.

Guaifenesin

Guaifenesin (3-(2-methoxyphenoxy)-1,2-propanediol) is a compound of the formula:

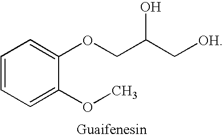

Guaifenesin is an expectorant that works by drawing water into the bronchi, thereby diluting and decreasing the viscosity of bronchial mucus. Thinning of pulmonary mucus makes it easier to clear from the lungs. By easing the clearing of phlegm from the lungs, guaifenesin reduces the frequency and forcefulness of coughing that is necessary to eliminate phlegm from the lungs.

When administered in a discrete dosage form and/or when co-administered with another ingredient, guaifenesin may be administered by any suitable route of administration, whether oral, rectal, parenteral, etc. However, in embodiments of the invention in which guaifenesin is administered in a dosage unit containing benzonatate, guaifenesin should be contained within a dosage unit that resists disintegration in the mouth, as discussed above in reference to benzonatate. Conveniently, guaifenesin may be administered in oral form, e.g. as an oral tablet, capsule, caplet, gel capsule, oral solution, syrup, suspension, etc. In particular embodiments, guaifenesin may be administered as an oral extended release tablet. In other embodiments, guaifenesin may be administered as an oral immediate release formulation.

The dosage of guaifenesin may vary from patient to patient. In some embodiments, guaifenesin is administered at a dosage of about 5 to about 3000 mg/day. Conveniently, guaifenesin may be administered in divided doses of about 5 to about 500 mg, from once to 10 times daily. In particular embodiments, e.g. when treating adults, the dosage will be about 100 mg/dose to 600 mg/dose, preferably about 200 mg/dose to about 400 mg/dose, every 2 to 6 hours, preferably about every 4 hours. The person skilled in the art will recognize that children are generally given doses lower than those used for adults. In some embodiments wherein the patient is a child of from 2 to 6 years of age, the dosage is about 25 mg/dose to about 200 mg/dose, preferably about 50 mg/dose to 100 mg/dose every 2 to 6 hours, preferably about every 4 hours. In some embodiments wherein the patient is a child of from about 6 to 12 years old, guaifenesin is administered at a dose of about 25 mg/dose to about 400 mg/dose, preferably about 100 mg/dose to about 200 mg/dose, from 2 to 6 times per day, preferably about 4 times per day. In some embodiments, guaifenesin may be prepared as a long-acting oral dose. In some embodiments wherein the oral dosage form is a long-acting oral dose, of from about 200 mg/dose to about 2400 mg/dose, preferably about 500 mg/dose to about 1500 mg/dose, in 2 to 4 doses administered every 6 to 24 hours, preferably about every 12 hours. In other embodiments, wherein the patient is a child of from about 2 to about 6 years of age, the oral dosage form is a long-acting oral dose of about 150 mg/dose to about 450 mg/dose, preferably about 300 mg, given in 2 to 4 doses every 6 to 24 hours, preferably about every 12 hours. In other embodiments, wherein the patient is a child of about 6 to about 12 years old, the extended release guaifenesin formulation is administered at a dose of about 300 mg/dose to about 900 mg/dose in 2 to 4 doses every 6 to 24 hours, preferably about every 12 hours or about 24 hours.

Dosage Unit

As used herein “dosage unit” means a discrete unit into which the dose of drug is divided. Such dosage units may include pills, tablets, caplets, capsules, gel caps, etc. In some embodiments of the invention, a dosage unit contains a therapeutically effective amount of benzonatate and guaifenesin, or a therapeutically acceptable salt thereof. In other embodiments of the invention, a dosage unit contains a fraction of a therapeutically effective amount of benzonatate and guaifenesin. In some particular embodiments, the fraction is about ½ to about ¼ of a therapeutically effective amount of the combination of benzonatate and guaifenesin. In some embodiments, benzonatate and guaifenesin are combined in an extended release dosage unit that provides a therapeutically effective amount of benzonatate and guaifenesin for from 8 to 24, preferably about 12 hours or about 24 hours.

In other embodiments, benzonatate and guaifenesin are formulated in separate dosage units. In some such embodiments, the dosage unit containing benzonatate is one that resists disintegration in the mouth, while the dosage unit containing guaifenesin is one that may or may not resist disintegration in the mouth. In some embodiments of the invention, the 2 discrete dosage units, one containing benzonatate and the other containing guaifenesin, together contain a therapeutically effective amount of benzonatate and guaifenesin, or a therapeutically acceptable salt thereof. In other embodiments of the invention, one, the other or both dosage units contain a fraction of a therapeutically effective amount of benzonatate or guaifenesin. In some particular embodiments, the fraction is about ½ to about ¼ of a therapeutically effective amount of the combination of benzonatate and guaifenesin. Thus, in some embodiments, each unit of benzonatate contains all, ½, ⅓ or ¼ of the amount of benzonatate which, when combined with the dose of guaifenesin, is therapeutically effective; and each unit of guaifenesin contains all, ½, ⅓ or ¼ of the guaifenesin which, when combined with the dose of benzonatate, is therapeutically effective. In some embodiments, benzonatate, guaifenesin or both are provided in extended release dosage units that provide a therapeutically effective amount of benzonatate, guaifenesin or both for from 8 to 24, preferably about 12 hours.

In particular embodiments, a therapeutically effective amount of benzonatate and guaifenesin is divided amongst 2 or more dosage units (1 or more of benzonatate and 1 or more of guaifenesin) and is contained within a package or kit. In some particular embodiments, the kit also includes instructions for administering the combination of benzonatate and guaifenesin to a patient to achieve a therapeutic effect. In more particular embodiments, the instructions include recommended dosing amounts and intervals, recommended maximum daily dosage, etc. In some particular embodiments, the effective dose of benzonatate and guaifenesin is divided among 1 to 4 containing benzonatate and 1 to 4 units containing guaifenesin. In more particular embodiments, the effective dose of benzonatate and guaifenesin is divided among 1 to 2 units containing benzonatate and 1 to 2 units containing guaifenesin. Other aspects of a kit according to the invention are described in more detail below.

Pharmaceutically Acceptable Salts

As benzonatate has a secondary amine in its structure, the person skilled in the art will recognize that benzonatate is capable of combining with at least one acid to form an acid-addition salt. In some preferred embodiments, benzonatate is combined with a pharmaceutically acceptable acid to form a pharmaceutically acceptable acid-addition salt. Although guaifenesin does not possess any functional groups that are ionized at or near a pharmaceutically acceptable pH, the person skilled in the art will recognize that the present invention provides guaifenesin and benzonatate, the latter of which is capable of forming a pharmaceutically acceptable salt. In crystalline or amorphous solid combinations of benzonatate and guaifenesin, the combination of guaifenesin and benzonatate are capable of forming, along with a suitable acid, a salt complex. In some embodiments, the term “pharmaceutically acceptable salts” refers to salts prepared from pharmaceutically acceptable non-toxic acids including inorganic or organic acids. In some embodiments, acid-addition salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Formulations

Effective doses of the guaifenesin and benzonatate, as described below, may be formulated in suitable pharmacological carriers and may be administered by any appropriate means, including, but not limited to orally or buccally. The combination of guaifenesin and benzonatate of the invention can be administered to a human patient by itself or in pharmaceutical compositions, where they are mixed with suitable carriers or excipients at doses effective to treat or mitigate cough. A therapeutically effective dose refers to that amount of the combination of guaifenesin and benzonatate sufficient to treat or mitigate cough.

Suitable formulations may include tablets, caplets or capsules for oral administration. In some embodiments, the formulations are extended release formulations comprising one or more extended release excipients, as will be discussed in more detail below. Such extended release Formulations may be formulated for twice-a-day (b.i.d.) or once-a-day (q.d.) administration. A suitable twice-a-day formulation will comprise approximately 600 to 1200 mg per dose of guaifenesin and about 200 to about 400 mg per dose of benzonatate. A suitable once-a-day formulation will comprise about 1200 to about 2400 mg per dose of guaifenesin and about 400 to about 800 mg per dose of benzonatate. A once-a-day formulation of guaifenesin and benzonatate will be formulated to release guaifenesin and benzonatate at such a rate that guaifenesin and benzonatate will be present in the body at a therapeutically effective level throughout a 24 hour period after administration of the dose to the patient. The formulation employed as an extended release formulation may be of the matrix, the osmotic pump or other known type of extended release formulation. The extended release dosage may be present in an single dosage form (tablet, caplet, capsule, etc.) or may be divided amongst two or more dosage forms; in either case the extended release formulations will release guaifenesin and benzonatate over an extended period of time so as to impart a therapeutic effect throughout a 12 or 24 hour period.

The pharmaceutical compositions containing the guaifenesin and benzonatate may be in a form suitable for oral use, for example, as tablets, troches, lozenges, suspensions, dispersible powders or granules, emulsions, hard or soft capsules, or syrups or elixirs. Compositions intended for oral use may be prepared according to any method known to those skilled in the art of manufacturing pharmaceutical compositions; and such compositions may contain one or more agents selected from the group consisting of sweetening agents, flavoring-agents, coloring agents and preserving agents in order to provide pharmaceutically palatable preparations. Tablets contain guaifenesin and benzonatate in admixture with non-toxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate buryrate may be employed.

Formulations for oral use may also be presented as hard gelatin capsules wherein guaifenesin and benzonatate are mixed with an inert solid diluent, for example, calcium carbonate, calcium phosphate or kaolin, or as soft gelatin capsules wherein the active ingredient is mixed with water soluble carrier such as polyethyleneglycol.

Syrups and elixirs may be formulated with sweetening agents, for example glycerol, propylene glycol, sorbitol or sucrose. Such formulations may also contain a demulcent, a preservative, flavoring and coloring agents and antioxidant.

As used herein, the term “immediate-release” refers to the substantially complete release of drug within a short time period following administration or initiation of dissolution testing, i.e., generally within a few minutes to about 1 hour.

A drug “release rate” refers to the quantity of drug released from a dosage form per unit time, e.g., milligrams of drug released per hour (mg/hr). Drug release rates for drug dosage forms are typically measured as an in vitro rate of dissolution, i.e., a quantity of drug released from the dosage form per unit time measured under appropriate conditions and in a suitable fluid. For example, dissolution tests can be performed on dosage forms placed in metal coil sample holders attached to a USP Type VII bath indexer and immersed in about 50 ml of acidified water (pH=3) equilibrated in a constant temperature water bath at 37° C. Aliquots of the release rate solutions are tested to determine the amount of drug released from the dosage form, for example, the drug can be assayed or injected into a chromatographic system to quantify the amounts of drug released during the testing intervals.

Unless otherwise specified, a drug release rate obtained at a specified time following administration refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 90% of drug within the dosage form has been released. This measurement is referred to as the “T₉₀” for the dosage form.

Unless specifically designated as “single dose” or at “steady-state,” the pharmacokinetic parameters disclosed and claimed herein encompass both single dose and steady-state conditions.

As used herein, the term “extended release” refers to the release of the drug from the dosage form over a period of many hours (e.g. 12 or 24 hours). Generally the extended release occurs at such a rate that blood (e.g., plasma) concentrations in the patient administered the dosage form are maintained within the therapeutic range, that is, above the minimum effective analgesic concentration or “MEAC” but below toxic levels, over a period of time of about 12 hours (b.i.d. administration) or 24 hours (q.d. administration).

Extended release dosage forms for twice daily oral dosing to a human patient for providing relief from cough are provided. In some embodiments, the extended release dosage form comprises an immediate release component and a sustained release component, wherein the immediate release component and the sustained release component collectively contain a therapeutically effective amount of a combination of guaifenesin and benzonatate.

In some embodiments, the extended release dosage form comprises an immediate release component and a sustained release component, which collectively contain a therapeutically effective amount of guaifenesin and benzonatate. In some embodiments, the extended release dosage form is adapted to provide a therapeutically effective dose of about 1200 to about 2400 mg of guaifenesin and about 400 to about 800 mg of benzonatate over a 24 hour period. In other embodiments, the extended release form is adapted to provide a therapeutically effective dose of about 600 to about 1200 mg of guaifenesin and about 200 to about 400 mg of benzonatate over a 12 hour period.

In some embodiments, a bilayer dosage form of guaifenesin and benzonatate is provided for twice daily oral administration to a human patient. Such a dosage comprises a drug layer comprising a therapeutically effective amount of guaifenesin and benzonatate, a nondrug layer comprising a high molecular weight polymer providing extended release of guaifenesin and benzonatate as an erodible composition upon imbibition of water, a semipermeable membrane providing a controlled rate of entry of water into the dosage form, and a flow promoting layer located between the drug layer and the semipermeable membrane. In some embodiments, such a dosage form provides about 600 to about 1200 mg of guaifenesin and about 200 to about 400 mg of benzonatate per dose.

In some embodiments, a bilayer dosage form of guaifenesin and benzonatate os provided for once daily oral administration to a human patient. Such a dosage comprises a drug layer comprising a therapeutically effective amount of guaifenesin and benzonatate, a nondrug layer comprising a high molecular weight polymer providing extended release of guaifenesin and benzonatate as an erodible composition upon imbibition of water, a semipermeable membrane providing a controlled rate of entry of water into the dosage form, and a flow promoting layer located between the drug layer and the semipermeable membrane. In some embodiments, such a dosage form provides about 1200 to about 2400 mg of guaifenesin and about 400 to about 800 mg of benzonatate per dose.

In some embodiments, the extended release component of the dosage form comprises: (1) a semipermeable wall defining a cavity and including an exit orifice formed or formable therein; (2) a drug layer comprising a therapeutically effective amount of guaifenesin and benzonatate contained within the cavity and located adjacent to the exit orifice; (3) a push displacement layer contained within the cavity and located distal from the exit orifice; (4) a flow-promoting layer between the inner surface of the semipermeable wall and at least the external surface of the drug layer that is opposite the wall; and the dosage form provides an in vitro rate of release of the guaifenesin and benzonatate for up to about 12 hours after being contacted with water in the environment of use. In some embodiments, such a dosage form provides about 600 to about 1200 mg of guaifenesin and about 200 to about 400 mg of benzonatate per dose.

In some embodiments, the extended release component of the dosage form comprises: (1) a semipermeable wall defining a cavity and including an exit orifice formed or formable therein; (2) a drug layer comprising a therapeutically effective amount of guaifenesin and benzonatate contained within the cavity and located adjacent to the exit orifice; (3) a push displacement layer contained within the cavity and located distal from the exit orifice; (4) a flow-promoting layer between the inner surface of the semipermeable wall and at least the external surface of the drug layer that is opposite the wall; and the dosage form provides an in vitro rate of release of the guaifenesin and benzonatate for up to about 24 hours after being contacted with water in the environment of use. In some embodiments, such a dosage form provides about 1200 to about 2400 mg of guaifenesin and about 400 to about 800 mg of benzonatate per dose.

In additional embodiments, the dosage form further comprises an immediate release component which preferably comprises a drug coating comprising a therapeutically effective amount of guaifenesin and benzonatate sufficient to provide an antitussive effect in a patient in need thereof. The drug coating provides an immediate release component to the dosage form providing the relatively immediate release and delivery of guaifenesin and benzonatate to the patient in need thereof.

Preparation of Osmotic Dosage Forms Containing a Guaifenesin and Benzonatate

The osmotic dosage form technology provides tunable sustained release dosage forms that can provide sustained release of one or more drugs, with or without the use of a drug coating providing immediate release of drug. Various types of osmotic dispensers are described in U.S. Pre-Grant Publication 2005/0158382, U.S. Pat. Nos. 3,845,770, 3,995,631, 4,034,756, 4,111,202, 4,320,759, 4,327,725, 4,449,983, 4,765,989, 4,940,465, and 6,368,626, all of which are incorporated herein by reference. The person skilled in the art will understand that the osmotic pumps may be adapted to delivery of the guaifenesin and benzonatate compositions according to the present invention through use of the appropriate dose of guaifenesin and benzonatate and osmotic dosage excipients.

Non-Osmotic Sustained Release Dosage Forms

In some embodiments, the invention makes use of other, non-osmotic extended release dosage forms. Commonly used oral controlled release dosage forms include matrix systems, osmotic pumps, and membrane controlled technologies.

Matrix Systems

Matrix systems are well known in the art. In a matrix system, the drug is homogenously dispersed in a release rate controlling matrix in association with conventional excipients. This admixture is typically compressed under pressure to produce a tablet. Drug is released from this tablet by diffusion and/or erosion. In a matrix system, a drug is incorporated into the polymer matrix by either particle or molecular dispersion. The former is simply a suspension of drug particles homogeneously distributed in the matrix, while the latter is a matrix with drug molecules dissolved in the matrix. Drug release occurs either by diffusion and/or erosion of the matrix system.

In a hydrophilic matrix, there are two competing mechanisms involved in the drug release: Fickian diffusional release and relaxational release. Diffusion is not the only pathway by which a drug is released from the matrix; the erosion of the matrix following polymer relaxation also contributes to the overall release. The relative contribution of each component to the total release is primarily dependent upon the properties of a given drug. For instance, the release of a sparingly soluble drug from hydrophilic matrices involves the simultaneous absorption of water and desorption of drug via a swelling-controlled diffusion mechanism. As water penetrates into a glassy polymeric matrix, the polymer swells and its glass transition temperature is lowered. At the same time, the dissolved drug diffuses through this swollen rubbery region into the external releasing medium. This type of diffusion and swelling generally does not follow a Fickian diffusion mechanism.

In a hydrophobic inert matrix system, the drug is dispersed throughout a matrix that involves essentially negligible movement of the device surface. For a homogeneous monolithic matrix system, the release behavior can be described by the Higuchi equation subject to the matrix-boundary conditions.

Drug release from a porous monolithic matrix system involves the simultaneous penetration of surrounding liquid, dissolution of drug, and leaching out of the drug through interstitial channels or pores. The volume and length of the openings in the matrix must be accounted for in a more complex diffusion equation. Thus, in contrast to the homogeneous monolithic matrix system, the release from a porous monolith is expected to be directly proportional to the drug concentration in the matrix.

The matrix formulations of this invention comprise guaifenesin, benzonatate and a pharmaceutically acceptable polymer. In some embodiments, the pharmaceutically acceptable polymer may be a water-soluble hydrophilic polymer, or a water insoluble hydrophobic polymer or nonpolymer waxes. Examples of suitable water soluble polymers include polyvinylpyrrolidine, hydroxypropylcellulose, hydroxypropylmethyl cellulose, methyl cellulose, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable waxes include fatty acids and glycerides.

In some embodiments, the polymer is selected from hydroxypropyl cellulose, hydroxypropylmethyl cellulose, and methyl cellulose. More preferably, the polymer is hydroxypropylmethyl cellulose.

The composition of the invention may also include pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, binders, etc. Such excipients are routinely used and are well known to those of skill in the art.

Diluents, or fillers, are added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc. Other diluents are routinely used and are well known to those of skill in the art.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc. Other lubricants are well known to those of skill in the art.

Glidants are also typically incorporated into the formulation. A glidant improves the flow characteristics of the granulation. Examples of glidants include talc, silicon dioxide, and cornstarch.

Binders may be incorporated into the formulation. Binders are typically utilized if the manufacture of the dosage form uses a granulation step. Examples of binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, antioxidants, or any other excipient commonly used in the pharmaceutical industry, etc. The amount of excipients used in the formulation will correspond to that typically used in a matrix system. The total amount of excipients, fillers and extenders, etc. varies.

Matrix formulations are generally prepared using standard techniques well known in the art. For example, they can be prepared by dry blending the polymer, filler, guaifenesin, benzonatate, and other excipients followed by granulating the mixture using an appropriate solvent until proper granulation is obtained. The granulation is done by methods known in the art. The wet granules are dried in a fluid bed dryer, sifted and ground to appropriate size. Lubricating agents are mixed with the dried granulation to obtain the final formulation.

The compositions of the invention can be administered orally in the form of tablets, pills, or the granulate may be loose filled into capsules. The tablets can be prepared by techniques known in the art and contain a therapeutically useful amount of guaifenesin and benzonatate and such excipients as are necessary to form the tablet by such techniques. Tablets and pills can additionally be prepared with enteric coatings and other release-controlling coatings for the purpose of acid protection, easing swallow ability, and controlling drug release, etc. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

Reservoir Polymeric Systems

In developing reservoir polymeric systems, commonly used methods include microencapsulation of drug particles, coating of tablets or multiparticulates, and press-coating of tablets. A polymeric membrane or press-coated layer offers a predetermined resistance to drug diffusion from the reservoir to the sink. The driving force of such systems is the concentration gradient of active molecules between reservoir and sink. In the case of film coating, the resistance provided by the membrane is a function of film thickness and characteristic of both the film as well as the migrating species in a given environment. Reservoir polymeric systems are well known to those of skill in the art.

The reservoir sustained release system of this invention comprises a therapeutically effective amount of guaifenesin, benzonatate and pharmaceutically acceptable polymer. The pharmaceutically acceptable polymer may be a hydrophobic polymer, a hydrophilic polymer or nonpolymer release rate-controlling materials. Examples of water hydrophilic polymers include polyvinylpyrrolidine, hydroxypropylcellulose, hydroxypropylmethyl cellulose, methyl cellulose, polyethylene glycol, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of water insoluble polymers include acrylates, cellulose derivatives such ethylcellulose or cellulose acetate, polyethylene, methacrylates, acrylic acid copolymers and high molecular weight polyvinylalcohols. Examples of suitable nonpolymer materials include fatty acids and glycerides, long carbon chain fatty acid esters, low molecular weight polyethylene.

In some embodiments, the release rate controlling polymer is often selected from ethylcellulose (Surelease from Colorcon, Aquacoat ECD from FMC), ammoniomethacrylate copolymers, methacrylic ester copolymers (Eudragit RL, RS, NE30D from Rohm America). The pore former in the membrane may be selected from hydroxypropyl cellulose, hydroxypropylmethyl cellulose, and polyethylene glycol. The amount of the polymer in the dosage form generally varies; and one of skill in the art will be able to determine the amount with routine experimentation.

The composition of the invention also typically includes pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, binders, etc. Such excipients are routinely used in the dosage forms of this invention.

Diluents, or fillers, are added in order to increase the mass of an individual dose to a size suitable for tablet compression. Diluents include powdered sugar, calcium phosphate, calcium sulfate, microcrystalline cellulose, lactose, mannitol, kaolin, sodium chloride, dry starch, sorbitol, etc.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of lubricants include talc, stearic acid, vegetable oil, calcium stearate, zinc stearate, magnesium stearate, etc.

Glidants are also typically incorporated into the formulation. A glidant improves the flow characteristics of the granulation. Examples of glidants include talc, silicon dioxide.

Binders may be incorporated into the formulation. Binders are typically utilized if the manufacture of the dosage form uses a granulation step. Examples of binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, plasticizers, antioxidants, or any other excipient commonly used in the pharmaceutical industry, etc. The amount of excipients used in the formulation will correspond to that typically used in a reservoir system. The total amount of excipients, fillers and extenders, etc. varies.

The reservoir formulations in the form of tablet or beads are generally prepared using techniques well known in the art. For example, tablet core are prepared by dry blending the filler, guaifenesin, benzonatate, polymer and other excipients followed by granulating the mixture using an appropriate solvent until proper granulation is obtained. The granulation is done by methods known in the art. The wet granules are dried in a fluid bed dryer, sifted and ground to appropriate size. Lubricating agents are mixed with the dried granulation to obtain the final formulation. The tablet can also be produced by dry granulation or direct compression. Beads used as substrates for coating are often prepared by extrusion/spheronization, use of non-peril seeds or granulation techniques.

Film coating of the tablets or beads with rate controlling polymers are performed using techniques well known in the art, such as pan coating or fluid-bed coating. Other coating techniques include compression coat using tableting machine. For example, to achieve proportional release of the guaifenesin and benzonatate of this invention, separate coating of guaifenesin and benzonatate may be performed followed by combining them into a single unit dosage form (tablet, capsule), or alternatively, partial coating of tablet core in the form of layered tablet are used. The reservoir system is also prepared by coating a matrix tablet core using film or press coating to provide dual control of drug release from the reservoir system.

The compositions of the invention can be administered orally in the form of tablets, pills, or the granulate may be loose filled into capsules. The tablets can be prepared by techniques known in the art and contain a therapeutically useful amount of guaifenesin, benzonatate and such excipients as are necessary to form the tablet by such techniques. Tablets and pills can additionally be prepared with enteric coatings and other release-modifying coatings for the purpose of acid protection, modified release, easing swallow ability, etc. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

Kits

In accordance with the present invention, benzonatate and guaifenesin may be combined in a single, discrete dosage unit or may be co-administered in separate, discrete dosage units, especially as part of a therapeutic kit. In some embodiments, the invention provides a kit comprising: a dosage unit comprising benzonatate; a dosage unit comprising guaifenesin; and optionally instructions for the use of the combination of benzonatate and guaifenesin for the treatment of coughing. In some currently preferred embodiments, the kit of the invention includes a dosage unit comprising benzonatate that is discrete from the dosage unit comprising guaifenesin. In some embodiments, the kit of the invention includes a dosage unit comprising benzonatate that contains about 50 to about 200 mg of benzonatate per dosage unit. Additionally, in some embodiments, the kit of the invention includes a dosage unit comprising guaifenesin contains about 50 to about 600 mg of guaifenesin per dosage unit. In some embodiments, the dosage unit comprising benzonatate is in the form of a tablet, a caplet, a capsule or a gel cap. In some embodiments, the comprising benzonatate is adapted to resist disintegration in the mouth and to dissolve readily in the stomach or intestines.

In embodiments in which benzonatate and guaifenesin are combined in an integrated dosage unit, the dosage form may be any form in which either benzonatate or guaifenesin is generally prepared, or may be a different dosage form than one in which either benzonatate or guaifenesin is generally prepared separately. In general, benzonatate is administered in an oral dosage form that ensures its delivery to the alimentary canal, specifically the stomach, without disruption of the dosage form. In particular, benzonatate is known to cause adverse reactions (numbness and potential choking) when applied directly to the mouth, and thus is delivered in an oral dosage form, such as a coated tablet, coated caplet, a gel capsule or other dosage form that will not easily dissolve in the mouth, but which will release benzonatate into the stomach or intestines. Thus a combination formulation of benzonatate and guaifenesin should preferably present the combination in a form that is not easily disrupted in the mouth or throat during swallowing of the dosage form. Such dosage forms include coated tablet, coated caplet, a gel capsule or other dosage form that will not easily dissolve in the mouth, but which will release benzonatate into the stomach or intestines upon administration of the dosage form to the patient.

In some embodiments, the invention provides a method of treating a cough, comprising co-administering to a patient a therapeutically effective amount of a composition comprising benzonatate, or a pharmaceutically acceptable salt thereof, and guaifenesin or a pharmaceutically acceptable salt thereof. In some embodiments, the pharmaceutical composition comprises benzonatate, or a pharmaceutically acceptable salt thereof, and guaifenesin, or a pharmaceutically acceptable salt thereof, is administered in an oral dosage form. In some embodiments, the oral dosage form is an oral capsule. In some embodiments a therapeutically effective daily dose of the combination of benzonatate and guaifenesin contains about 5 mg/day to about 1000 mg/day of benzonatate and about 5 mg/day to about 3000 mg/day of guaifenesin. In some embodiments, the therapeutically effective amount of the combination of benzonatate and guaifenesin is divided into 2 to 8, preferably 2 to 4 doses containing about 50 mg/dose to about 400 mg/dose of benzonatate and about 100 mg/dose to about 600 mg/dose of guaifenesin, each. In some more particular embodiments, the therapeutically effective amount of the combination of benzonatate and guaifenesin is divided into 2 to 8, preferably 2 to 4 doses containing about 100 mg/dose to about 200 mg/dose of benzonatate and about 200 mg/dose to about 400 mg/dose of guaifenesin, each. In some embodiments, the oral dosage form is adapted to deliver to a patient, in particular a pediatric patient, about 10 mg to about 100 mg of benzonatate and about 50 mg to about 400 mg of guaifenesin per dose. In some embodiments, the patient is sensitive to opiate-derived antitussives or dextromethorphan. In some embodiments, patient is a pediatric patient.

In some embodiments, the invention provides a method of treating congestion of the lung, comprising co-administering to a patient a therapeutically effective amount of a composition comprising benzonatate, or a pharmaceutically acceptable salt thereof, and guaifenesin or a pharmaceutically acceptable salt thereof. In some embodiments, the pharmaceutical composition comprises benzonatate, or a pharmaceutically acceptable salt thereof, and guaifenesin, or a pharmaceutically acceptable salt thereof, is administered in an oral dosage form. In some embodiments, the oral dosage form is an oral capsule. In some embodiments, the pharmaceutical composition is adapted to provide a dose of about 50 mg to about 400 mg of benzonatate and about 100 mg to about 600 mg of guaifenesin per dose. In some embodiments, the pharmaceutical composition provides approximately 100 mg to 200 mg of benzonatate and about 200 mg to about 400 mg of guaifenesin per dose. In some embodiments, the oral dosage form is adapted to deliver to a patient, in particular a pediatric patient, about 10 mg to about 100 mg of benzonatate and about 50 mg to about 400 mg of guaifenesin per dose.

In some embodiments, the invention provides a method of simultaneously treating lung congestion and cough, comprising administering to a patient a therapeutically effective amount of a composition comprising benzonatate and guaifenesin. In some embodiments, the composition comprises benzonatate, or a pharmaceutically acceptable salt thereof, and guaifenesin, or a pharmaceutically acceptable salt thereof, is administered in an oral dosage form. In some embodiments, the oral dosage form is an oral capsule. In some embodiments, the pharmaceutical composition is adapted to provide a dose of about 50 mg to about 400 mg of benzonatate and about 100 mg to about 600 mg of guaifenesin per dose. In some embodiments, the pharmaceutical composition provides approximately 100 mg to 200 mg of benzonatate and about 200 mg to about 400 mg of guaifenesin per dose. In some embodiments, the oral dosage form is adapted to deliver to a patient, in particular a pediatric patient, about 10 mg to about 100 mg of benzonatate and about 50 mg to about 400 mg of guaifenesin per dose. In some embodiments, the patient is sensitive to opiate-derived antitussives or dextromethorphan. In some embodiments, the patient is a pediatric patient.

### EXAMPLE 1

### Combination Dosing of Benzonatate and Guaifenesin

A cohort of about 4 to 400 patients of age 20 to 60 is divided into 4 groups. The first, benzonatate only group is given benzonatate at a dose of about 50 to 200 mg every 4 to 12 hours. The second, guaifenesin only group, is administered guaifenesin at a does of about 200 mg to about 400 mg per does every 2 to 6 hours. The third, combination group (co-administration of benzonatate and guaifenesin) is given benzonatate at a dose of about 50 to 200 mg and guaifenesin 200 mg to about 400 mg per does every 2 to 8 hours. The fourth, control group is given a placebo every 2 to 8 hours. The response of each group to its respective dosing regimen is determined by objective (e.g. observed number of coughs per hour) or subjective (patient's perceived frequency and/or severity of coughing) measures and the results are tabulated. In general, a combination of benzonatate and guaifenesin provides superior relief of cough as compared to benzonatate or guaifenesin therapy alone.

Although preferred embodiments of the present invention have been shown and described herein, it will be apparent to those skilled in the art that such embodiments are provided by way of example only. Numerous variations, changes, and substitutions will be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered herein.

What is claimed is:

1. A pharmaceutical composition comprising a therapeutically effective combination of benzonatate and guaifenesin, or a pharmaceutically acceptable salt thereof.

2. The pharmaceutical composition of claim 1, wherein the pharmaceutical composition is an orally available dosage unit.

3. The pharmaceutical composition of claim 1, wherein the pharmaceutical composition is formulated for once-per-day (q.d.) dosing.

4. The pharmaceutical composition of claim 3, wherein the pharmaceutical composition is formulated to deliver about 400 to 800 mg of benzonatate and about 1200 to about 2400 mg of guaifenesin per dose.

5. The pharmaceutical composition of claim 1, wherein the pharmaceutical composition is formulated for twice-per-day (b.i.d.) dosing.

6. The pharmaceutical composition of claim 5, wherein the pharmaceutical composition is formulated to deliver about 200 to about 400 mg of benzonatate and about 600 to about 1200 mg of guaifenesin per dose.

7. The pharmaceutical composition of claim 1, wherein the pharmaceutical composition is an oral capsule, tablet, caplet or gel cap.

8. The pharmaceutical composition of claim 1, wherein the pharmaceutical composition is adapted to provide a dose of about 50 mg to about 400 mg of benzonatate and about 100 mg to about 600 mg of guaifenesin per dosage unit.

9. A method of treating a cough, congestion or both, comprising administering to a patient suffering from a cough a therapeutically effective amount of a composition comprising benzonatate, or a pharmaceutically acceptable salt thereof, and guaifenesin.

10. The method of claim 9, wherein the composition comprising benzonatate, or a pharmaceutically acceptable salt thereof, and guaifenesin is administered in an oral dosage form.

11. The method of claim 10, wherein the oral dosage form is an oral tablet, caplet, capsule or gel cap.

12. The method of claim 9, wherein the therapeutically effective amount of the composition is administered once-per-day.

13. The method of claim 12, wherein the therapeutically effective amount of guaifenesin is about 1200 to about 2400 mg and the therapeutically effective amount of benzonatate is about 400 to about 800 mg.

14. The method of claim 9, wherein the therapeutically effective amount of the composition is administered twice-per-day.

15. The method of claim 14, wherein the therapeutically effective amount of guaifenesin is about 600 to about 1200 mg and the therapeutically effective amount of benzonatate is about 200 to about 400 mg.

16. The method of claim 9, wherein the oral dosage form is adapted to deliver about 50 mg to about 400 mg of benzonatate and about 100 mg to about 600 mg of guaifenesin per dose.

17. The method of claim 9, wherein the oral dosage form is adapted to deliver about 100 mg to about 200 mg of benzonatate and about 200 mg to about 400 mg of guaifenesin per dose.

18. The method of claim 9, wherein the patient is sensitive to opiate-derived antitussives or dextromethorphan.

19. The method of claim 9, wherein the patient is a pediatric patient.

20. The method of claim 19, wherein the composition comprising benzonatate, or a pharmaceutically acceptable salt thereof, and guaifenesin is administered in an oral dosage form.

21. The method of claim 20, wherein the oral dosage form is an oral tablet, caplet, capsule or gel cap.

22. The method of claim 19, wherein the therapeutically effective amount of the composition is administered on a once-per-day basis.

23. The method of claim 22, wherein the therapeutically effective amount of guaifenesin is about 150 to about 1200 mg per dose.

24. The method of claim 22, wherein the therapeutically effective amount of benzonatate is about 50 to about 200 mg per dose.

25. The method of claim 19, wherein the therapeutically effective amount of the composition is administered on a twice-per-day basis.

26. The method of claim 25, wherein the therapeutically effective amount of guaifenesin is about 300 to about 600 mg per dose.

27. The method of claim 25, wherein the therapeutically effective amount of benzonatate is about 50 to about 100 mg per dose.

28. A method of treating congestion of the lung, comprising co-administering to a patient a therapeutically effective amount of benzonatate and guaifenesin, wherein the therapeutically effective amount of benzonatate and guaifenesin comprises a first discrete dosage unit comprising benzonatate or a pharmaceutically acceptable salt thereof and a second discrete dosage unit comprising guaifenesin.

29. The method of claim 28, wherein the first dosage unit does not contain guaifenesin.

30. The method of claim 28, wherein the second dosage unit does not contain benzonatate.

31. The method of claim 28, wherein the first dosage unit, the second dosage unit, or both the first and second dosage units are adapted for oral administration

32. The method of claim 28, wherein the first dosage unit, the second dosage unit, or both the first and second dosage units are in the form of an oral tablet, caplet, capsule or gel cap.

33. The method of claim 28, wherein the first dosage unit and the second dosage unit together are adapted to deliver about 50 mg to about 400 mg of benzonatate or a bioequivalent amount of a pharmaceutically acceptable salt of benzonatate and about 100 mg to about 600 mg of guaifenesin per dose.

34. The method of claim 28, wherein the first dosage unit and the second dosage unit together are adapted to deliver about 200 to about 400 mg of benzonatate or a bioequivalent amount of a pharmaceutically acceptable salt thereof and about 600 to about 1200 mg of guaifenesin per dose.

35. The method of claim 34, wherein the dosage forms are adapted for twice-per-day dosing.

36. The method of claim 28, wherein the first dosage unit and the second dosage unit together are adapted to deliver about 400 to about 800 mg of benzonatate or a bioequivalent amount of a pharmaceutically acceptable salt thereof and about 1200 to about 2400 mg of guaifenesin per dose.

37. The method of claim 36, wherein the dosage forms are adapted for once-per-day dosing.

38. The method of claim 28, wherein the first dosage unit and the second dosage unit together are adapted to deliver about 100 mg to about 200 mg of benzonatate or an equivalent amount of a pharmaceutically acceptable salt of benzonatate and about 200 mg to about 400 mg of guaifenesin per dose.

39. The method of claim 28, wherein the first dosage unit is adapted to resist disintegration in the mouth.

40. The method of claim 39, wherein the first dosage unit is in an immediate release or an extended release form.

41. A kit comprising:
(a) a dosage unit comprising benzonatate;
(b) a dosage unit comprising guaifenesin; and optionally
(c) instructions for the use of the combination of benzonatate and guaifenesin for the treatment of coughing.

42. The kit of claim 41, wherein the dosage unit comprising benzonatate is discrete from the dosage unit comprising guaifenesin.

43. The kit of claim 41, wherein the dosage unit comprising benzonatate contains about 50 to about 800 mg of benzonatate per dosage unit.

44. The kit of claim 41, wherein the dosage unit comprising guaifenesin contains about 50 to about 2400 mg of guaifenesin per dosage unit.

45. The kit of claim 41, wherein the dosage unit comprising benzonatate is in the form of a tablet, a caplet, a capsule or a gel cap.

46. The kit of claim 45, wherein the dosage unit comprising benzonatate is adapted to resist disintegration in the mouth and to dissolve readily in the stomach or intestines.

47. The kit of claim 41, wherein the dosage units are adapted for once-per-day dosing.

48. The kit of claim 47, wherein the kit is adapted for pediatric dosing.

49. The kit of claim 48, wherein the kit is adapted to deliver about 50 to about 200 mg of benzonatate or a bioequivalent amount of a pharmaceutically acceptable salt thereof and about 600 to about 1200 mg of guaifenesin per dose.

50. The kit of claim 47, wherein the kit is adapted to deliver about 400 to about 800 mg of benzonatate or a bioequivalent amount of a pharmaceutically acceptable salt thereof and about 1200 to about 2400 mg of guaifenesin per dose.

51. The kit of claim 41, wherein the dosage units are adapted for twice-per-day dosing.

52. The kit of claim 51, wherein the kit is adapted for pediatric dosing.

53. The kit of claim 52, wherein the kit is adapted to deliver about 50 to about 200 mg of benzonatate or a bioequivalent amount of a pharmaceutically acceptable salt thereof and about 600 to about 1200 mg of guaifenesin per dose.

54. The kit of claim 51, wherein the kit is adapted to deliver about 200 to about 400 mg of benzonatate or a bioequivalent amount of a pharmaceutically acceptable salt thereof and about 600 to about 1200 mg of guaifenesin per dose.

55. The kit of claim 41, wherein the dosage units are adapted for q.i.d. dosing.

56. The kit of claim 55, wherein the kit is adapted to deliver bout 50 to about 400 mg of benzonatate or a bioequivalent amount of a pharmaceutically acceptable salt thereof and about 100 to about 600 mg of guaifenesin per dose.
